(19) United States
(12) Patent Application Publication
(10) Pub. No.: US 2025/0134427 A1
(43) Pub. Date: May 1, 2025

(54) DEVICES, SYSTEMS, AND METHODS FOR ANALYTE DETECTION USING NON-ENZYMATIC SENSORS

(71) Applicant: NERv Technology Inc., Kitchener (CA)

(72) Inventors: Mohamed Okasha, Kitchener (CA); Vivek Maheshwari, Kitchener (CA)

(21) Appl. No.: 18/934,721

(22) Filed: Nov. 1, 2024

Related U.S. Application Data
(60) Provisional application No. 63/595,093, filed on Nov. 1, 2023.

(51) Int. Cl. A61B 5/1486; A61B 5/00; A61B 5/145; A61B 5/15; A61B 5/157

(52) U.S. Cl. CPC A61B 5/1486; A61B 5/14532; A61B 5/14546; A61B 5/150992; A61B 5/157; A61B 5/7275

(57) ABSTRACT

The present disclosure describes various embodiments of devices, systems, and methods for analyte detection using non-enzymatic sensors. In one embodiment, there is disclosed sensor device for detecting one or more analytes, the sensor device comprising: one or more working electrodes, each of the one or more working electrodes comprising a plurality of reduced or oxidized nanocomposite chains having metal domains deposited on a substrate of each of the one or more working electrodes; a reference electrode and a counter electrode, connected, by a circuit, to the one or more working electrodes.

### FIELD OF THE DISCLOSURE

The present disclosure relates generally to sensor devices, systems, and methods, and more particularly, to devices, systems, and methods for analyte detection using non-enzymatic sensors.

### BACKGROUND

Surgical procedures may use open and minimally invasive techniques on users, such as patients, in order to identify and treat pathological conditions or improve body functions. Surgeries performed due to a variety of reasons have an inherent risk of post-operative complications such as hemorrhages, infections and leakages to develop.

One of the most dangerous complications for surgery is a complication known as anastomotic leakage. Anastomotic leakage may develop after an anastomosis is performed where two organs are surgically connected, and is most commonly observed in gastrointestinal surgery. Anastomotic leakage leads to luminal contents leaking into the peritoneal cavity which may cause a cascade of deadly complications to arise. This typically involves a form of severe sepsis, peritonitis, morbidity and it may lead to mortality.

Using traditional techniques, it can take three to seven days on average for a leak to be diagnosed. This is very dangerous especially considering that every hour of delay causes a considerable increase in the morbidity and mortality risk for the patient.

Each year, 70 million major abdominal surgery (MAS) procedures are performed globally. MAS includes pancreatic, hepatobiliary, and colorectal surgeries with a primary anastomosis. These surgeries have a complication rate of 30-60%, 20% of which have significant detrimental effects that require invasive treatments and enhanced patient monitoring. The occurrence of such complications can have dire consequences, both acute and long-term, including a significant degree of morbidity and mortality for affected patients.

Post surgical complications may further comprise, without limitation, hemorrhage, post-operative leakages, ischemia, infection, and sepsis. Early detection of these complications, and subsequent intervention, is a key factor in reducing mortality

Typically, medical facilities wait for clinical factors such as abdominal pain, fever and tachycardia to arise before diagnosis. Existing technologies for detection of post-operative complications, such as anastomotic leakage, may be nonspecific, inefficient, time-consuming, expensive, and/or lacking in the ability to provide real-time detection of the complication.

Diagnosis may require ineffective and costly CT scans, MRIs, blood tests and nurse checkups.

Continuously monitoring patients after a surgery for one or more biomarkers which may indicate a post-surgical complication may be a more effective and efficient method of detecting complications than current methods.

One such biomarker may comprise lactate, which is the conjugate base of lactic acid, a hydroxy monocarboxylic anion and is produced as a byproduct from anaerobic respiration.

Ischemia, which is a condition that arises when there is an inadequate supply of blood to an organ or part of the body, results in a lack of blood supply to cells, thereby causing anaerobic respiration and lactate as the biproduct. Early detection of a post-surgical complication such as an anastomotic leak (AL), may be detected by monitoring lactate in a biofluid. For example, in blood, the normal lactate concentration is within a range 0.5-2 mM.

Studies shown correlation between peritoneal lactate levels and anastomotic leak diagnosis. If there is a sharp increase in lactate in the blood, this could be an indication of a lack of, or reduced amount, of oxygen in the blood, indicating that organs are at risk, there has been a leakage, or both. When tissue at the anastomosis is ischemic, the tissue at the anastomosis may die, the tissue walls allowing content to leak.

Bini et al. (Bini, R.; Ferrari, G.; Aprà, F.; Viora, T.; Leli, R.; Cotogni, P. Peritoneal lactate as a potential biomarker for predicting the need for reintervention after abdominal surgery. J. of Trauma and Acute Care Surgery. 2014, 77 (2), 376-380.) showed that peritoneal fluid with lactate levels of greater than 9.1 mM had an AL positive predictive value of 89% with a sensitivity of 81.9% and specificity of 82%. Peritoneal lactate also responds more quickly to changes in oxygen, than does blood/serum.

Another biomarker may comprise glucose, a simple monosaccharide. Monitoring biofluids for decreases in glucose may provide early detection of anistomotic leakage. Following a leak, decreased oxygen in the blood supply from ischemia would result in decreased glucose. Alternatively, decrease in glucose may indicate consumption of glucose by the cells during anaerobic respiration, where glucose is not being replenished due to ischemia.

Similarly, monitoring peritoneal fluids for decreasing glucose levels may indicate a complication. Normal glucose peritoneal concentration is within a range of 4-5 mM.

Currently, glucose and lactate are generally detected with enzyme-based sensors, measuring electron transfer through enzyme-catalyzed processes involving the target analyte. For example, lactate detection typically monitors the enzymatic reactions lactate oxidase or lactate dehydrogenase.

Although enzymatic sensors have good specificity and sensitivity, and can detect lactate in biological pH conditions, they tend to degrade quickly, making them improper candidates for continuous monitoring applications. Enzymatic sensors are most commonly available as test strips for one time use. Work is in progress to produce longer lasting sensors, but enzyme stability has been the primary limiting factor towards achieving this goal.

Enzymes can be unstable and are prone to denaturation, especially over longer periods of use, making them unsuitable for long term or multiple day measurements, such as those conducted by in-line biofluid monitoring devices (i.e. those disclosed in U.S. patent application Ser. No. 17/598,118).

Research has been made in controlled environments and mostly in non isotonic conditions. These metal and metal oxide catalysts are also utilized for other applications such as hydrogen and water evolution, and catalytic fuel cells.

This background information is provided to reveal information believed by the applicant to be of possible relevance. No admission is necessarily intended, nor should be construed, that any of the preceding information constitutes prior art or forms part of the general common knowledge in the relevant art.

### BRIEF SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

To date, there are no devices or technologies specifically designed for the real-time continuous measurement and monitoring analytes in biological/isotonic pH conditions, which are traditionally detected via enzymatic catalysis. There may exist therefore, a need for systems and devices which may continuously, and in real-time, measure and monitor a plurality of analytes. There may further be a need for computer implemented methods of processing and analyzing sensor data relating to a plurality of analytes.

In accordance with an embodiment, there is disclosed a sensor device for detecting one or more analytes, the sensor device comprising: one or more working electrodes, each of the one or more working electrodes comprising a plurality of reduced or oxidized self-assembled nanocomposite chains having metal domains deposited on a substrate of each of the one or more working electrodes; a reference electrode and a counter electrode, connected by a circuit, to the one or more working electrodes.

In some embodiments, each working electrode is configured to measure, via a catalytic reaction between one or more analytes and the reduced or oxidized nanocomposite chains having metal domains, one or more electrochemical signals correlating to a concentration of the one or more analytes.

In some embodiments, the sensor device further comprises one or more of: polymers or aptamers, functionalized to a surface of the reduced or oxidized nanocomposite chains.

In some embodiments, the metal domains comprise a template and one or more metal ions, the template functionalized by negatively charged capping agents.

In some embodiments, the template is a gold nanoparticle template, and the negatively charged capping agents are citrate ions.

In some embodiments, the one or more metal ions are one or more of: a transition metal and a noble metal.

In some embodiments, the one or more metal ions are selected from a group comprising: Platinum, Nickel, Cobalt, Copper, Manganese, Iridium, Iron, Vanadium, Ruthenium, and Rubidium.

In some embodiments, the sensor device is fluidically coupled to biofluids of a patient for the measurement of the one or more electrochemical signals correlating to the one or more analytes in the biofluids of the patient.

In some embodiments, the one or more analytes comprise at least one of glucose or lactate.

In some embodiments, the sensor device is configured to operate in isotonic and physiological pH conditions.

In accordance with another embodiment, there is provided a method for detecting and monitoring one or more analytes, the method comprising: measuring, via a catalytic reaction between the one or more analytes and the plurality of reduced or oxidized nanocomposite chains having metal domains deposited on one or more working electrodes of a sensor device, one or more electrochemical signals of the one or more analytes.

In some embodiments, the method further comprises: receiving at a server, via the sensor device, the one or more electrochemical signals; separating, via a multiple analyte analysis model, the one or more electrochemical signals; correlating each of the one or more electrochemical signals to a concentration of each of the one or more analytes; and generating an output based on the concentration of each of the one or more analytes.

In some embodiments, the sensor device is fluidically coupled to biofluids of a patient, for measuring the one or more electrochemical signals relating to the one or more analytes in the biofluids of the patient.

In some embodiments, the one or more electrochemical signals is measured continuously and in real-time.

In some embodiments, the one or more analytes comprises at least one of glucose or lactate.

In some embodiments, the measurement of the one or more electrochemical signals is conducted in isotonic and physiological pH conditions.

In some embodiments, the one or more analytes are identified, via the multiple analyte analysis model, based on oxidation potential or reduction potential of the one or more analytes.

In some embodiments, the output comprises a post-surgical complication risk assessment of the patient.

In accordance with another embodiment, there is provided a non-transitory, computer readable storage medium, the computer-readable storage medium including instructions, that, when executed, performs the steps of: receiving at a server, via a sensor device, one or more electrochemical signals; separating, via a multiple analyte analysis model, the one or more electrochemical signals; correlating each of the one or more electrochemical signals to a concentration of each of the one or more analytes; and generating an output based on the concentration of each of the one or more analytes.

In some embodiments, the one or more analytes comprises at least one of glucose or lactate.

Other aspects, features and/or advantages will become more apparent upon reading of the following non-restrictive description of specific embodiments thereof, given by way of example only with reference to the accompanying drawings.

### BRIEF DESCRIPTION OF THE DRAWINGS

To easily identify the discussion of any particular element or act, the most significant digit or digits in a reference number refer to the figure number in which that element is first introduced.

Several embodiments of the present disclosure will be provided, by way of examples only, with reference to the appended drawings, wherein:

FIG. 1A illustrates a schematic diagram of a method for preparing template-metal nanocomposite chains: gold-metal nanocomposite chains, in accordance with one embodiment;

FIG. 1B illustrates experimental data relating to Transmission Electron Microscopy (TEM) and Electron Energy Loss Spectroscopy (EELS) images of gold-platinum (Au—Pt) nanocomposite chains, in accordance with one embodiment;

FIG. 2A illustrates a schematic diagram of a method for preparing segregated domain template-metal nanocomposite chains: gold-metal nanocomposite chains, with two different metals, in accordance with one embodiment;

FIG. 2B illustrates a schematic diagram of a method for preparing homogenously distributed domain template-metal nanocomposite chains: gold-metal nanocomposite chains, with two different metals, in accordance with one embodiment;

FIG. 3 illustrates experimental data relating to TEM and EELS imagery of a hybrid nanocomposite chain: Gold-Ni:Pt nanocomposite chains, in accordance with one embodiment;

FIG. 4 illustrates experimental data relating to an X-ray Diffraction spectrum (XRD) of a hybrid nanocomposite chain: Gold-Ni:Pt nanocomposite chains, in accordance with one embodiment;

FIG. 5A illustrates a schematic diagram relating to the drop-cast method of depositing nanocomposite chains on an electrode, in accordance with various embodiments;

FIG. 5B illustrates a schematic diagram relating to the drop-cast method of depositing nanocomposite chains on an electrode in an existing sensor device, in accordance with various embodiments;

FIG. 5C illustrates a schematic diagram relating to the screen printing method of depositing nanocomposite chains on an electrode, in accordance with various embodiments;

FIG. 5D illustrates a schematic diagram relating to the vacuum filter method of depositing nanocomposite chains on an electrode, in accordance with various embodiments;

FIG. 6 illustrates a schematic diagram of a sensor device having nanocomposite chains for detection of one or more bioanalytes, in accordance with one embodiment;

FIG. 7A illustrates a plot showing current vs. lactate concentration, in the determination of sensitivity of a hybrid nanocomposite chain: 1:2 Nickel-Platinum Gold (Au 1:2 Ni:Pt), tested in 2X PBS buffer (similar to physiological pH), in accordance with one embodiment;

FIG. 7B illustrates a plot showing current vs. time, in order to demonstrate real-time electrochemical lactate detection of a hybrid nanocomposite chain: Au 1:2 NiPt, in accordance with one embodiment;

FIG. 8A depicts cyclic voltammetry (CV) curves showing Au—Pt nanoparticle chains oxidizing lactic acid, in accordance with one embodiment;

FIG. 8B depicts cyclic voltammetry (CV) curves showing Ni—Au nanoparticle chains oxidizing lactic acid, in accordance with one embodiment;

FIG. 9 depicts plots showing current vs. lactate concentration, in order to demonstrate changes in sensitivity accompanying changing ratios of catalyst/co-catalyst-in this case, Ni/Pt, in accordance with one embodiment;

FIG. 10A illustrates a chronoamperometry (CA) plot showing current at 1.44V vs. time for Ni AuNP nanocomposite chains, in order to demonstrate the changes in sensor sensitivity to lactate when adding cations to nanocomposite chains, in accordance with one embodiment;

FIG. 10B illustrates a chronoamperometry (CA) plot showing current at 1.44V vs. time for 1:2 Ni:Pt oxidized and reduced pre-assembled AuNP nanocomposite chains, in order to demonstrate the changes in sensor sensitivity to lactate when adding cations to nanocomposite chains, in accordance with one embodiment;

FIG. 11A illustrates the real-time detection capabilities of 1:2 Ni:Pt nanocomposite chains, using chronoamperometry (CA), in accordance with one embodiment;

FIG. 11B illustrates the relative sensitivity of 1:10 Ni:Pt to various analytes, in accordance to one embodiment;

FIG. 12A illustrates experimental data relating to copper-gold (Cu—Au) nanoparticle chains EELS mapping showing the structure, in accordance with one embodiment;

FIG. 12B illustrates experimental data relating to real-time detection capabilities of Cu-Au NP chains through Chronoamperometry (CA), in accordance with one embodiment;

FIG. 13 illustrates plots showing the sensitivity of a 1:1 Cu: Pt—Au hybrid nanocomposite chain, in accordance with one embodiment;

FIG. 14 illustrates plots showing calibration curves for determining multiple analytes with a hybrid nanocomposite chain, in accordance with one embodiment;

FIG. 15 illustrates a schematic diagram of the non-enzymatic sensor device, in accordance with one embodiment;

FIG. 16 illustrates a flow chart relating to methods of monitoring multiple analytes with a non-enzymatic sensor device, in accordance with one embodiment;

FIG. 17A illustrates a differential pulse voltammetry (DPV) curves using a hybrid nanocomposite chain comprising AuHEA 1/2 PtNiCuCoV, in accordance with one embodiment;

FIG. 17B illustrates a differential pulse voltammetry (DPV) plot showing current vs. lactate concentration in the determination of sensitivity of a hybrid nanocomposite chain AuHEA 1/2 PtNiCuCoV, in accordance with one embodiment;

FIG. 17C illustrates a differential pulse voltammetry (DPV) plot showing current vs. run number, in accordance with one embodiment;

FIG. 18A illustrates a full electrical circuit connecting a plurality of working electrodes, a reference electrode, and a counter electrode, in accordance with one embodiment; and

FIG. 18B illustrates a part of the electrical circuit connecting a plurality of working electrodes, a reference electrode, and a counter electrode housed in the sensor device assembly, in accordance with one embodiment.

Elements in the several drawings are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

The following disclosure may, in part, provide a sensor device comprising non-enzymatic materials such as metals and metal oxides to act as a catalyst and co-catalyst for continuous bioanalyte monitoring using electrochemical methods.

A sensor device comprising several metals and metal oxides may preferably be used to electro-oxidize and continuously detect lactate in isotonic pH conditions.

In some embodiments, the bioanalyte(s) being monitored may comprise lactate, glucose, or some combination thereof. In some embodiments, the bioanalytes being monitored may also comprise small biological analytes such as dopamine other hormones.

The sensor device may be produced via a nanoparticle self-assembly method, wherein one or more metals and/or metal oxides are added to a solution comprising nanoparticles capped with negatively charged species, and left to self-assemble into nanoparticle-metal chains.

The method may further comprise a reduction step. The method may be carried out at room temperature, with no extra heating/mixing/degassing steps. The method may further comprise an electrode preparation step, wherein the reduced nanoparticle-metal/metal oxide chains are deposited onto an electrode.

Methods of depositing the nanoparticle-metal/metal oxide chains onto an electrode or any flexible substrate, may include any methods known in the art, including, but not limited to, drop casting, filtration, and screen printing. Electrodes may be glass. The nanoparticle chains may be deposited onto an existing sensor device, which may be connected inline with a patient's biofluid for monitoring bioanalytes in the patient's biofluid.

The method may further comprise adding a binding agent, such as a polymer, in order to strengthen adsorbance of the nanopoarticle-metal/metal oxide chains to the electrode and/or to the sensor device. The binding agent may, for example, comprise Nafion™, or other synthetic/halogenated polymers that are commercially available.

In one embodiment, the method comprises combining several metals and/or metal oxides such as platinum, copper and nickel oxide etc. to form a catalyst and co catalyst surface on the nanoparticles.

Several metals and metal oxides are capable of electro-oxidizing and continuously detecting lactate and/or glucose in isotonic pH conditions.

Materials such as metals and metal oxides are more stable than enzymatic materials, allowing them to be used for long-term continuous sensory applications.

Using gold nanoparticles as a template for the self-assembly process may allow the combination of several metal and metal oxides onto the gold nanoparticle surface as catalysts and co-catalysts with ability to change elemental ratios and distributions of material allowing the creation of the unique catalytic surface for the electrochemical detection of a specific analyte.

Systems, methods, and devices disclosed herein relate to continuous lactate and/or glucose detection in isotonic biological pH range using a unique combination of catalysts and co-catalysts.

Systems, methods, and devices disclosed herein relate to the development and methods of using stable, non-enzymatic, solid-state sensors. Electrocatalytic material surface design is important for target analyte adsorbing/desorbing.

Currently, glucose and lactate are generally detected with enzyme-based sensors, measuring electron transfer through enzyme-catalyzed processes involving the target analyte. For example, lactate detection typically monitors the enzymatic reactions lactate oxidase or lactate dehydrogenase.

Lactate reaction mechanism is likely a 2 or more-step process:
1. Adsorption onto catalyst surface;
   a. (intermediate forms)
2. Removal of hydrogen & subsequent oxidation and desorption of resulting pyruvic acid from the catalyst's surface.

The mechanism may differ between isotonic pH and those studied in literature-for example, in isotonic pH, pyruvic acid may or may not be a final product.

Lactate detection in isotonic pH, as well as integrating glucose detection in a lactate sensor, has significant advantages over prior art methods. Use of machine learning to differentiate between signals may provide further advantages.

According to the disclosure, there are disclosed methods of continuously monitoring lactic acid, for example, after surgery for the early detection of ischemia and anastomotic leaks post surgery. By monitoring trends of lactic acid (and lactate/glucose ratio), it can be possible to detect ischemia and some forms of anastomotic leaks earlier than conventional methods.

Systems and methods disclosed herein may be integrated into existing sensor devices, including sensor devices fluidically inline with patient biofluids, such as embodiments disclosed in international patent application PCT/CA2020/050395, which is herein incorporated by reference, in its entirety.

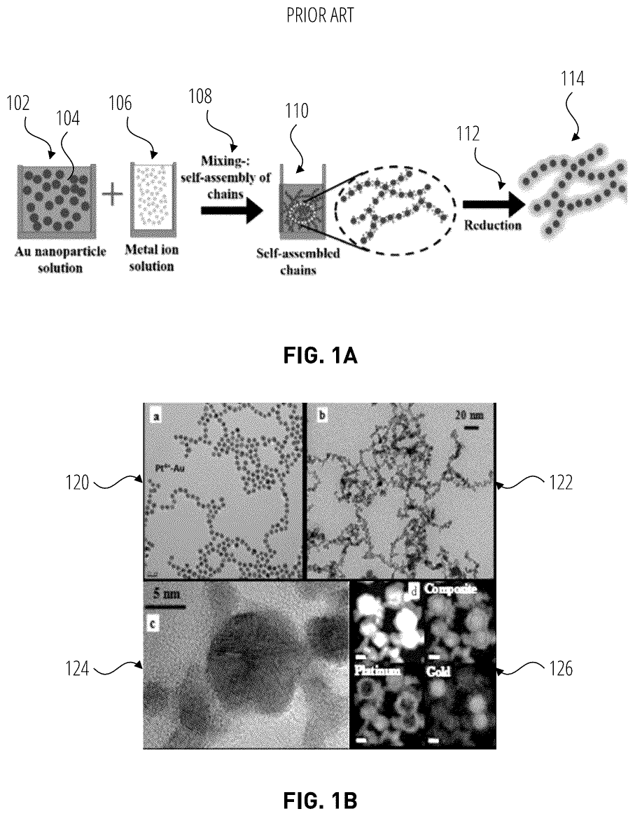

FIG. 1A illustrates methods for preparing nanocomposite chains from the prior art.

The present disclosure relates to preparing non-enzymatic sensors using self-assembled nanocomposite chains, such as those from Pu, L.; Fan, H.; Maheshwari, V. Formation of Microns Long Thin Wire Networks With Controlled Spatial Distribution of Elements. Catal. Sci. Technol. 2020, 10, 2020-2028.

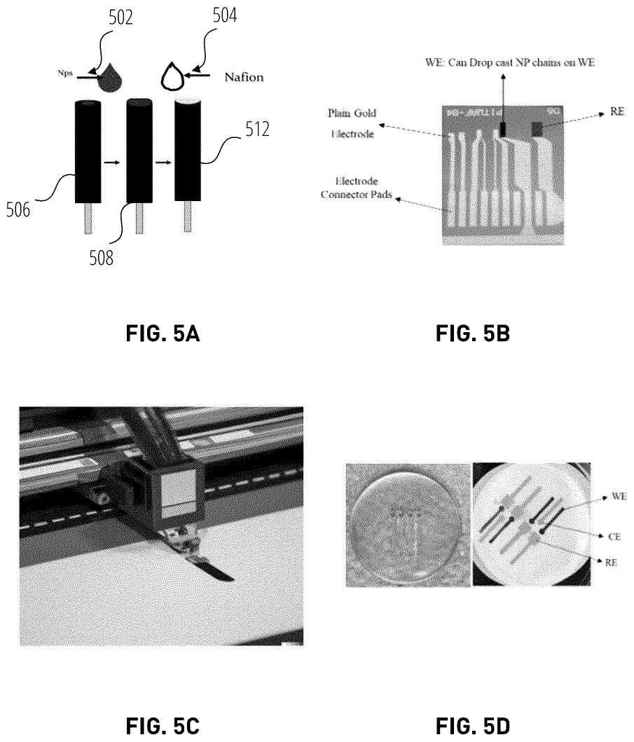

The present disclosure teaches methods of preparing non-enzymatic sensors using self-assembled nanoparticles. The methods may comprise:
1. A solution of a nanoparticle template 102 capped or functionalized by negatively charged capping agents 104 is mixed with a metal ion solution 106;
2. A plurality of template-metal nanocomposite chains 110 (chains comprising template surrounded by metal cations), form in-solution through self-assembly 108;
3. Reduce and/or oxidize 112 chains with a reducing/oxidizing agent in order to convert metal cations to metallic state and/or to metal hydroxides/metal oxides;
4. Reduced/oxidized nanocomposite chains deposited on an electrode surface, flexible substrate, or on an existing sensor assembly (shown in more detail in FIG. 5A).

The nanocomposite chains 114, 110 formed from these methods have a high exposed surface area to volume ratio, and thus a high contact surface area (SA) for a target analyte (for example, glucose and lactate). High contact SA requires less catalytic material than prior art sensors composed of bulk metal catalysts. Using less catalytic material for sensor preparation is advantageous, as the cost of material is lowered.

The present disclosure teaches the use of citrate capped gold nanoparticles for use as a template. These are commercially available, for example, through Sigma Aldrich. Citrate capped gold nanoparticles have a negatively charged surface due to the citrate ions present. In one embodiment, the template nanoparticles may have a diameter of 10-12 nm each. When a metal cation is added to the nanoparticle solution, the positively charged cations self assemble around the negatively charged nanoparticles. The self assembled nanocomposites form chain-like structures, which may be micron-long chains composed of up to hundreds of the template nanoparticles, resulting in chains which may have a length from approximately 100 nm, to lengths in the micron range. It should be readily understood that other nanoparticle templates and anion capping agents may be used in order to form nanocomposite chains with a high contact surface area.

The nanoparticle chains are deposited on an electrode/substrate/sensor device to form a sensor device. The size and surface area of the sensor device may vary, and is customizable based on the amount of nanoparticle chains deposited on the device itself. The sensor de vice could be small (in the nano or micron range), for deposition in or on a patient catheter, or a portable sensor device. In some embodiments, the sensor device could be large enough for use in a benchtop analytical instrument.

Several different metal cations can be introduced to the solution, which allows the mixing of elements on the gold nanoparticle surface, with different spatial distributions and elemental ratios depending on how the nanocomposite was prepared. The chains can be reduced to reduce the metal cation to its metallic state, or oxidized to form metal oxides or hydroxides. The relative ratios and time of cation addition can change the atomic structure and distribution of the catalyst surface allowing the formation of several unique surface structures.

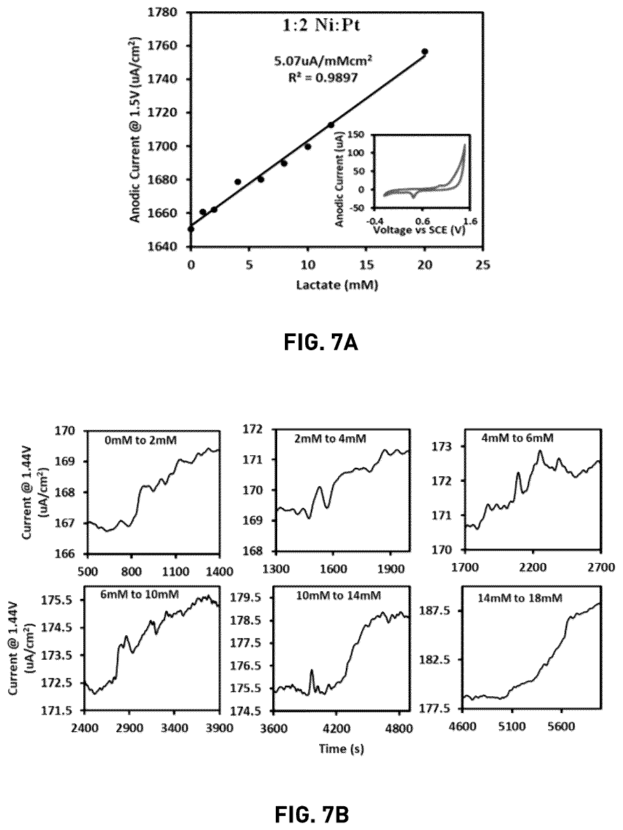

The nanocomposites may be employed in conjunction with a reference and counter electrode on a sensor device, to detect changes in lactate continuously using chronoamperometry (CA) or pulse voltammetry techniques. Lactate detection has been achieved in a PBS buffer solution at a measuring potential around 1.44V using platinum and oxides of nickel, and has been achieved in even lower potentials using a combination of platinum and copper (shown in greater detail in FIG. 7A).

Using gold nanoparticles as a template to form self assembled nanocomposites containing a mixture of the catalyst and co-catalyst is what makes lactate detection possible in biological pH conditions primarily due to synergistic effects of the combined elements. Since the materials do not denature, they are stable enough to be used for continuous monitoring in biological applications.

Since non-enzymatic catalysts are not always specific towards the analyte being detected as opposed to enzymatic sensors, methodologies such as molecular imprinting for lactate can be deployed to prevent other similarly sized analytes from interacting with the catalyst surface. Polymeric material, imprinted with the shape of lactate, may be deployed on a sensor device or used as functional groups on the nanoparticle chains, acting as an artificial enzyme, and increasing the sensor's selectivity towards lactate. Moreover, the ability to incorporate lactate specific ligands/aptamers may also increase sensor sensitivity and specificity.

Furthermore, the ability to add and combine multiple catalysts and co catalysts (metal ions) can be utilized to create multiple analyte sensors. The proposed lactate sensor can be used to detect glucose in tandem with lactate, if glucose oxidation is achieved at a different potential than that of lactate. This can potentially produce a single sensor that can switch between detecting glucose and lactate, as well as other small/similar analytes. The metals in the metal ion solution 106 may comprise: Platinum, Nickel, Cobalt, Copper, Manganese, and Iridium, Iron, and Vanadium, Ruthenium, and Rubidium, and the like. Oxidation states may vary—for example, both Iron (II) and Iron (III) may be employed. Many other early transition metals and noble metals can potentially be used with this method

The following are examples of methods of preparing gold-metal cation nanocomposite chains. Relative and specific amounts may be adjusted as-needed to change composition and performance of the sensors.

### Example of Au-NiPt Sample Preparation:

1. 140 ul of 4.5 mg/ml PtCl₄ added per 1ml of 10-12 nm Au nanoparticle (NP) solution. Left to pre-assemble;
2. 60 ul of 4 mg/ml NiCl₂ added per 1 ml 10-12 nm Au NP solution. Left to pre-assemble;
3. Pre-assembled chains mixed to form either 1:2 Ni:Pt or 1:10 Ni:Pt;
4. Sample treated with NaOH and H₂O₂ to oxidize Ni, then NaBH₄ to reduce Pt.

### Examples of Au—CuPt Sample Preparation:

1. 70 ul of 4.5 mg/ml PtCl₄ added per 1 ml of 10-12 nm AuNP soln. and left to pre-assemble several days, then 30 ul of 4 mg/ml CuCl₂ added and soln. left to assemble. Sample reduced using NaBH₄.
2. 70 ul of 4.5 mg/ml PtCl₄ added per 1 ml of 10-12 nm AuNP soln. and 30 ul of 4 mg/ml CuCl₂ added at same time and soln. left to assemble. Sample reduced using NaBH₄.
3. 30 ul of 4 mg/ml CuCl₂ added per 1 ml of 10-12 nm AuNP soln. and left to pre-assemble several days, then 70 ul of 4.5 mg/ml PtCl₄ added, and soln. left to assemble. Sample reduced using NaBH₄. Cation concentration of chains form 1:1 Cu:Pt.

Relative amounts can be changed.

### Examples of High Entropy Alloy (HEA) Sample Preparation: Au-PtCuCoNiV HEA

1. 140/5 ul of 4.5 mg/ml PtCl₄ added per 1 ml of 10-12 nm AuNP soln. and left to pre-assemble several days, then 60/5 ul of 4 mg/ml CuCl₂, 60/5 ul of 4 mg/ml CoCl₂, 60/5 ul of 4 mg/ml NiCl₂ 70/5 ul of 4 mg/ml VCl₃ added and soln. Left to assemble. Sample reduced using NaBH₄.
2. 140/5 ul of 4.5 mg/ml PtCl₄ added per 1 ml of 10-12 nm AuNP soln. and 60/5 ul of 4 mg/ml CuCl₂, 60/5 ul of 4 mg/ml CoCl₂, 60/5 ul of 4 mg/ml NiCl₂ 70/5 ul of 4 mg/ml VCl₃ added at same time and soln. Left to assemble. Sample reduced using NaBH₄.
3. 60/5 ul of 4 mg/ml CuCl₂, 60/5 ul of 4 mg/ml CoCl₂, 60/5 ul of 4 mg/ml NiCl₂ 70/5 ul of 4 mg/ml VCl₃ added per 1 ml of 10-12 nm AuNP solution and left to pre-assemble several days, then 70 ul of 4.5 mg/ml PtCl₄ added, and solution left to self-assemble. Sample reduced using NaBH₄.
4. Cation conc. of chains form 1:1:1:1:1 Pt:Cu:Co:Ni:V.

It may be preferable, when preparing nanocomposite chains comprising one or more metals, to adjust relative amounts of cation solutions added, in order to manipulate the ratios of cations mixed in solution and therefore making up the nanocomposite chain. For example, in a one-metal nanocomposite chain made of Platinum cations and gold nanoparticles, 140 uL of 4.5 mg/mL PtCl₄ per 1 ml AuNP may be added. If another cation were desired, in a 1:1 ratio with Pt, the concentration of Pt may be decreased in order to achieve this ratio.

For example, for the preparation of a 5 cation HEA as described above, amounts/concentrations of cation solutions are expressed as being divisible by 5, such that the ratio of cations in solution is 1:1:1:1:1. Adding multiple solutions of cations without diluting them, or decreasing the amount added, may result in aggregation or precipitation of the gold nanoparticles.

Relative amounts of cations can be changed.

### Sample Preparation Considerations:

Samples containing a metal and metal oxide may need a reducing and oxidizing agent respectively. The order of oxidation and reduction of sample may affect performance.

Pt chains take a much longer time to self-assemble (6 to 8 days) vs smaller cations like Cu, Co, Ni, V (1 to 2 days) due to relative size and thermodynamics. Since assembly time can affect surface distribution, timing of adding ions should be taken into consideration.

FIG. 1B illustrates a transmission electron microscopy (TEM) image of: A) assembled Pt⁴⁺ on Gold nanoparticle (Au NP) template-metal nanocomposite chains 110 (120), B) reduced Pt and Au nanocomposites 114 (122), C) high resolution TEM (HRTEM) showing a gold nanoparticle surrounded by Pt (124), and D) Electron Energy Loss Spectroscopy (EELS) elemental mapping of the reduced chain structures (126).

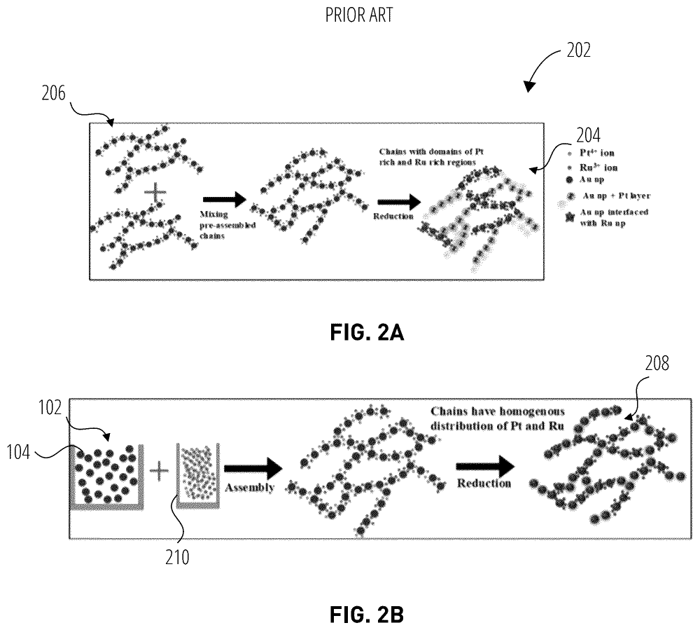

FIG. 2A illustrates methods for preparing hybrid nanocomposite chains 202, from the prior art.

The present disclosure relates to preparing non-enzymatic sensors using self-assembled hybrid nanocomposite chains, such as those from Pu, L.; Fan, H.; Maheshwari, V. Formation of Microns Long Thin Wire Networks With Controlled Spatial Distribution of Elements. Catal. Sci. Technol. 2020, 10, 2020-2028.

The present disclosure teaches methods of preparing non-enzymatic sensors using self-assembled nanoparticles. In this embodiment, hybrid nanocomposite chains-segregated domains 204 comprise a plurality of different cations, formed from two or more solutions of pre-assembled chains 206. Two or more solutions of pre-assembled chains each comprising one or more differing metal cations (formed by the methods described in FIG. 1A), are mixed to form hybrid nanocomposite chains—segregated domains 204 having the one or more differing metal cations of each of the two or more solutions of pre-assembled chains 206.

Upon mixing the pre-assembled chains 206, the chains 206 form hybrid nanocomposite chains-segregated domains 204, and are then reduced and/or oxidized 112, in order to form metallic or metal oxide hybrid nanoparticle chains. The reduced and/or oxidized hybrid nanoparticle chain 204 may be deposited on an electrode to form a hybrid nanoparticle-based non-enzymatic sensor which may be capable of detecting one or more target analytes.

The method described and illustrated in FIG. 2A may result in hybrid nanocomposite chains-segregated domains 204—the pre-assembled chains 206 do not typically rearrange to form new chains with a homogeneous distribution of cations—rather, they assemble around each other in solution to result in alternating cation chains, each cation chain a segregated cation domain. The resulting structure is highly dependent on the metal combination chosen.

Alternatively, FIG. 2B shows another method of forming hybrid nanocomposite chains 202.

As in FIG. 1A, a solution comprising a nanoparticle template 102, the template capped/functionalized by a negatively charged capping agent 104, is mixed with a metal ion solution. In this embodiment, the metal ion solution is a mixed ion solution 210, having two or more different kinds of cations dissolved in solution.

Upon mixing the nanoparticle template 102 solution with the mixed ion solution 210, the metal cations self-assemble 108 to form hybrid nanocomposite chains—homogeneous distribution 208. The resulting nanocomposite chains 208 may then be oxidized and/or reduced in order to derive the metallic/oxidized product which may be deposited on a sensor which may be capable of detecting one or more target analytes.

Generally, the method described and illustrated in FIG. 2B results in hybrid nanocomposite chains—homogeneous distributions 208—the cations are relatively homogeneously distributed amongst the nanoparticle chains. The resulting structure is highly dependent on the choice of cations, as well as the timing of introducing cations to solution. For example, Pt and Ru are both noble metals with similar lattice spacings, and therefore may form homogeneously distributed alloys fairly easily.

Hybrid-gold nanoparticle chains can create structures with several metal/metal oxides while controlling spatial distribution of elements. Varying degrees of homogeneity and spatial distribution result in differing analyte detection capabilities. Nanocomposite chains 114 and hybrid nanocomposite chains 202 may be customized to the exact detection requirements of a sensor.

The exact structure of the nanoparticle chains may vary, and can be customized based on the desired use of the nanoparticle chains. The chains may comprise heterogeneous metal domains or alloys (multiple metals in a domain). The metal domains may repeat within the chain length. The surface can be heterostructure or homogenous, depending on method of preparation and cation constituents.

Methods of developing nanocomposite chains may further comprising adding one or more of: surface polymer or aptamers to a surface of the oxidized or reduced nanocomposite chains, the resulting structure preferably having greater specificity/selectivity to the target analytes. Preferably, a sensor comprising nanocomposite chains with polymeric/aptameric functional groups, may resultingly have less exposure to biomolecules that are not the target analyte, resulting in less noise in a measured signal. Molecularly imprinted polymers may be added to the surface of the reduced/oxidized nanoparticle chains, the molecularly imprinted polymers sized and configured to only allow desired analyses such as lactate and glucose to reach and interact with, the catalyst surface.

The metals in the mixed ion solution 210 may comprise: Platinum, Nickel, Cobalt, Copper, Manganese, and Iridium, Iron, and Vanadium, Ruthenium, and Rubidium, and the like. Oxidation states may vary—for example, both Iron (II) and Iron (III) may be employed. Many other early transition metals and noble metals can potentially be used with this method

In addition, some chains, such as Au—Cu (without Pt) or Au—Ni (without Pt), may break apart upon reduction. Addition of Pt or other co-catalysts may contribute to stabilizing the chains, resulting in more metallic Cu and Ni forming on the on the nanocomposite chain.

Using a plurality of metals results in the ability to control multi-element catalyst spatial arrangements. A non-enzymatic sensor with both a catalyst and co-catalyst surfaces, may be developed using these methods, each of the catalyst and co-catalyst being capable of reacting with varying analytes for detection (for example, both glucose and lactate).

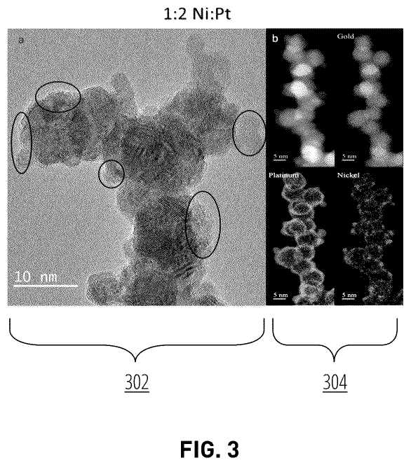

FIG. 3 illustrates HRTEM image of A) 1:2 Ni:Pt AuNP pre-assembled nanocomposite chains, Ni domains circled 302 and B) EELS elemental mapping of the chains structure 304.

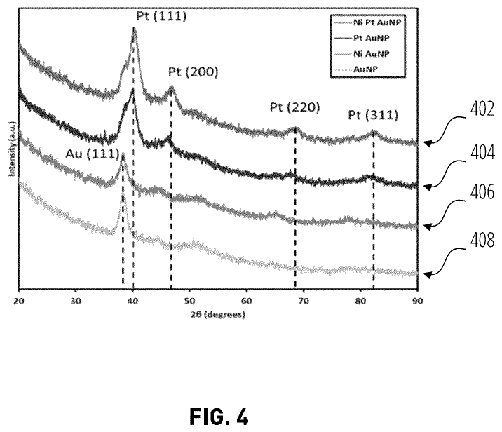

FIG. 4 shows x-ray diffraction (XRD) patterns of nanoparticle (NP) chains and hybrid nanocomposite chains: AuNPs, reduced Ni AuNPs, reduced Pt AuNPs, and reduced 1:3 Ni:Pt AuNP nanocomposite chains. Pt and Au peaks are labelled.

The resolved fringes are those corresponding with the Pt shell.

The unresolved fringes correspond with smaller surrounding Ni domains, indicating that the Ni is amorphous.

Varying surface distribution of catalysts/co-catalysts may affect performance of a sensor.

FIG. 5A, FIG. 5B, FIG. 5C, and FIG. 5D illustrate various methods of preparing a non-enzymatic sensor according to various embodiments.

Preparing an electrode for a non-enzymatic sensor may comprise one or more different methods.

Since nanoparticles are liquid nanoparticle chains 502, they may be deposited on an electrode 506 by various methods: dropcast (FIG. 5A, FIG. 5B), screen printed using an inkjet printer (FIG. 5C), or vacuum filtered on a polycarbonate filter (FIG. 5D), or any other method of electrode preparation known in the art. The electrode 506 may comprise glassy carbon. Screen printing may comprise altering the viscosity of liquid nanoparticle chains 502 to form “ink”.

### EXAMPLE

### Electrode Preparation for Lactate (LA) Detection

1. Samples 502 drop cast (15 ul) onto glassy carbon electrode 506 for electrochemical analysis to form nanocomposite electrode 508.
2. nanocomposite electrode 508 coated with binding agent 504 to support nanoparticles adhesion to electrode 506, forming a nanocomposite working electrode 512;
3. Electrodes 512 are conditioned for 150 cycles using Cyclic Voltammetry (CV);
4. Solutions stirred at 250 rpm.

The binding agent 504 may comprise Nafion™, or other synthetic/halogenated polymers that are commercially available.

Lactate may be detectible through various electrochemical methods, where the nanocomposite electrode 508 constitutes a working electrode. Some electrochemical methods may comprise cyclic voltammetry (CV), Chronoamperometry (CA), potentiometry, and the like.

As seen in FIG. 5B, the nanoparticle chains may be deposited onto an existing sensor device, which may be connected inline with a patient's biofluid for monitoring bioanalytes in the patient's biofluid. The existing sensor device may have sensing/working electrodes in addition to the nanocomposite electrode 508.

In FIG. 5D, WE, CE and RE represent working electrode, counter electrode, and reference electrode, respectively.

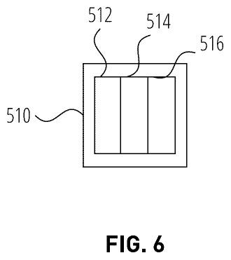

FIG. 6 illustrates a schematic of a non-enzymatic sensor device 510.

A non-enzymatic sensor device 510 may have 3-electrodes-the nanocomposite working electrode 512, connected to a counter electrode 514, and a reference electrode 506, by a circuit.

The sensor device may 510 is amperometric, and in an embodiment, has a 3-electrode system. The working electrode 512 is composed of the sensing material (nanocomposite chains), where the primary reaction occurs. The sensor device 510 may further comprise a reference electrode 516 (for example, Ag/AgCl, and the like), and a counter electrode. The illustrated embodiment is amperometric (voltage is applied, current is then measured) but other sensing mechanisms can also apply, including, but not limited to, impedimetric and potentiometric methods.

A sensor device for measuring and monitoring multiple analytes may comprise one or more working electrodes 512. For example, several different nanoparticle chains 114 may be deposited on one working electrode 512 for multiple analyte detection. Alternatively, the sensor device 510 may comprise a plurality of working electrodes 512, each with a different nanoparticle chain deposited on each of the plurality of working electrodes, and each of the working electrodes being connected to the circuit having the counter and reference electrode.

An advantage of using AuNP nanocomposite chains is that, since the material itself is conductive, it can be used to directly create the working/interfacing electrode, but can also be added on top of other working electrodes.

FIG. 7A illustrates sensitivity of a hybrid nanocomposite chain: Au-NP 1:2 Ni:Pt, tested in 2X PBS buffer (similar to physiological pH).

The hybrid nanocomposite chain demonstrated ability to oxidize LA in isotonic pH due to synergistic effects.

FIG. 7B illustrates results of electrochemical lactate detection of a hybrid nanocomposite chain: Au-NP 1:2 NiPt.

The plots illustrate anodic currents vs lactate concentrations at an oxidation potential of 1.5V vs SCE for 1:2 Ni:Pt and a close-up look at the real-time LA detection using CA at an oxidation potential of 1.44V vs SCE for 1:2 Ni:Pt.

Current changes were observed observed between 1.44-1.5V, and sensitivity was found to be approximately 1 uA/mMcm².

When nickel and platinum are combined, lactic acid may be detected in neutral pH conditions. Increasing the loading density, by adding more cations to the nanocomposite chains may increase sensitivity in a sensor device comprising a working electrode formed from nanocomposite chains.

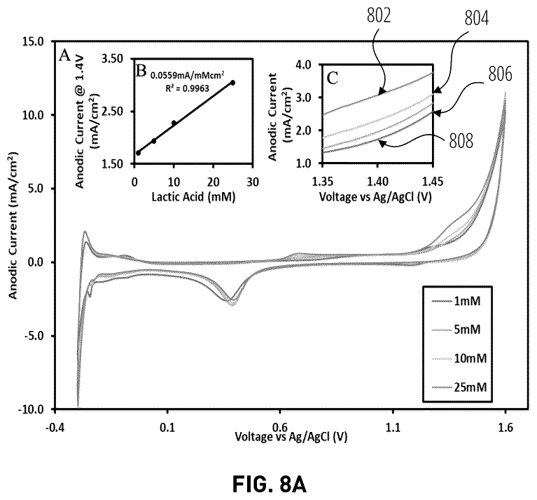

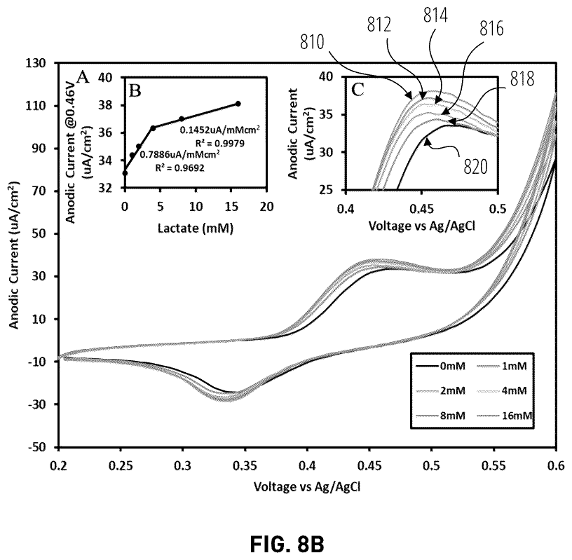

FIG. 8A depicts cyclic voltammetry (CV) curves showing platinum-gold nanoparticle chains oxidizing lactic acid.

Bulk platinum is a catalyst that has been shown to oxidize lactate in acidic conditions, in accordance with results measured by Sedenho et al. (Sedenho, G. G. C.; Lee, P. T.; Toh, H. S.; Salter, C.; Johnston, C.; Stradiotto, N. R.; Compton, R. G. The Electro-Oxidation of Lactic Acid at Platinum Microparticles and Polycrystalline Platinum Electrode. Int. J. of Electrochem. Sci. 2016, 11, 2166-2176). However, bulk Pt has a relatively low surface area compared to nanocomposite chains, and thus more bulk material than nanocomposite material may be required in order to achieve a similar degree of oxidation between the two.

The CV curve of Platinum-gold nanoparticle (Pt AuNP) self-assembled chains is illustrated with different lactate concentrations in 0.5M H₂SO₄ (A). The plotted anodic currents vs lactate concentrations at an oxidation potential of 1.4V (B), and CV cycle close-up (C).

By comparing these results to literature results, it can be seen that the nanocomposite material performs similarly to the bulk material. Platinum oxidizes lactic acid in 0.5M sulfuric acid at around 1.4V.

FIG. 8B depicts cyclic voltammetry (CV) curves showing nickel-gold nanoparticle chains oxidizing lactic acid.

Bulk nickel/nickel oxide is a catalyst that has been shown to oxidize lactate in basic conditions, in accordance with results measured by Kim et al (Kim, S.; Kim, K.; Kim, H. J.; Lee, H. N.; Park, T. J.; Park, Y. M. Non-Enzymatic Electrochemical Lactate Sensing by NiO and Ni(OH)2 Electrodes: A Mechanistic Investigation. Electrochimica Acta. 2018, 276, 240-246). Bulk Ni has a relatively low surface area compared to nanocomposite chains, and thus more bulk material may need to be used compared to the nanocomposite material in order to achieve a similar degree of oxidation.

Nickel oxide and nickel hydroxide are able to detect lactate in basic conditions using sodium hydroxide and potassium chloride, with the detection happening at a potential of approximately 0.5V.

The nickel-gold nanoparticle chains oxidized lactic acid in sodium hydroxide at 0.45V, showing that both of these individual nanoparticle chains, performed similarly to their bulk material counterparts.

Testing Au Pt chains, and Au Ni chains in acidic and basic conditions, respectively, resulted in platinum oxidizing lactic acid in 0.5M sulfuric acid at ˜1.4V, and nickel oxidizing lactic acid in sodium hydroxide at ˜0.45V, indicating that both of these individual nanoparticle chains may perform similarly to their bulk material counterparts.

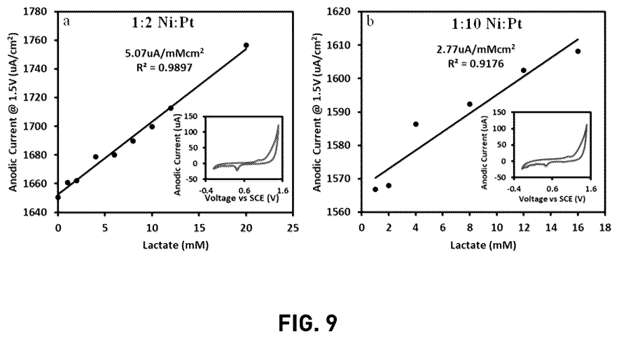

FIG. 9 illustrates the change in sensitivity accompanying changing ratios of catalyst/co-catalyst-in this case, Ni/Pt.

The plotted anodic currents vs lactate concentrations at an oxidation potential of 1.5V vs SCE for 1:2 Ni:Pt (A) and 1:10 Ni:Pt (B) oxidised and reduced pre-assembled AuNP nanocomposite chains in a 2X PBS solution. Insets are their respective second CV cycles in all plotted lactate concentrations.

Current changes are observed at 1.5V, and 1:2 Ni:Pt was shown to be more sensitive to LA changes than 1:10 Ni:Pt and calibration curves had better linearity.

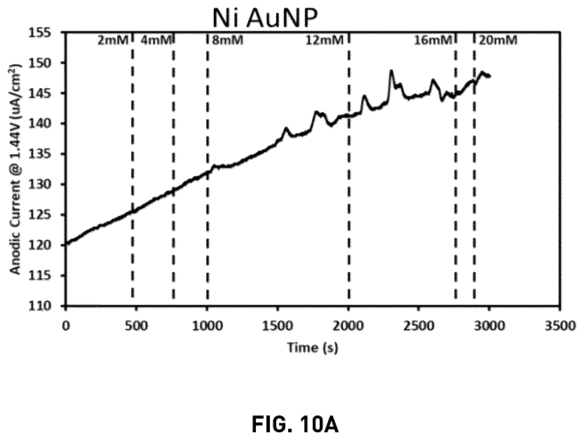

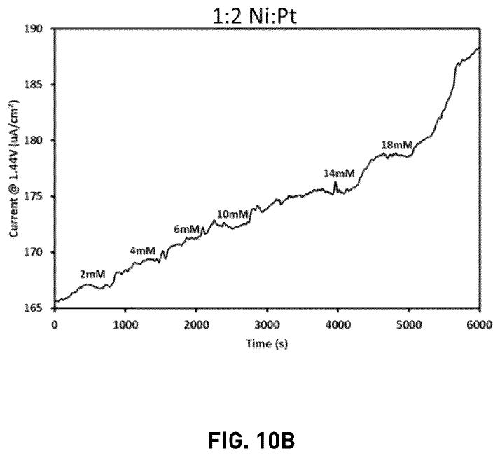

FIG. 10A illustrates real-time LA detection using CA at an oxidation potential of 1.44V vs SCE for Ni AuNP nanocomposite chains in a 2X PBS solution. Each vertical line shows LA addition.

FIG. 10B illustrates real-time LA detection using CA at an oxidation potential of 1.44V vs SCE for 1:2 Ni:Pt oxidised and reduced pre-assembled AuNP nanocomposite chains in a 2X PBS solution.

For Ni AuNP shown in FIG. 10A, little-to-no real-time response was observed upon increasing lactate concentration. However, real-time response was observed for 1:2 Ni:Pt, as shown in FIG. 10B with each LA addition. It can therefore be seen, that combining Ni and Pt allows for real-time LA detection.

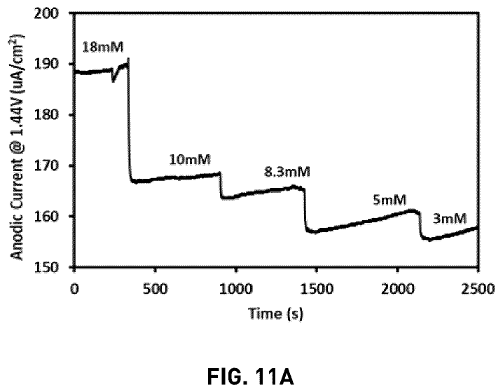

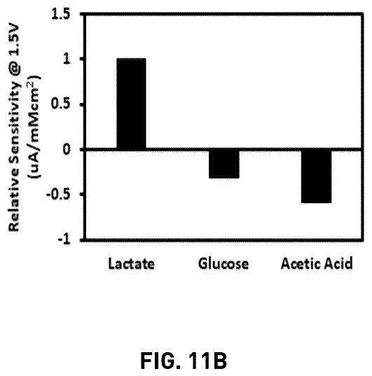

FIG. 11A illustrates the real-time detection capabilities of 1:2 Ni:Pt nanocomposite chains, using chronoamperometry (CA).

In the illustrated embodiment, real-time detection of continuously decreasing (through dilution), lactate, is achieved with a 1:2 Ni:Pt reduced self-assembled nanocomposite chains. This is measured in a 2x PBS buffer solution (similar to biological pH).

Relative ratios of Ni and Pt may be adjusted in order to change the sensitivity of the sensor. In addition, the incorporation of other catalytic materials into the nanoparticle chains, by the methods illustrated and described in FIG. 1A and FIG. 2A and FIG. 2B.

FIG. 11B is a plot illustrating the relative sensitivity of 1:10 Ni:Pt to various analytes.

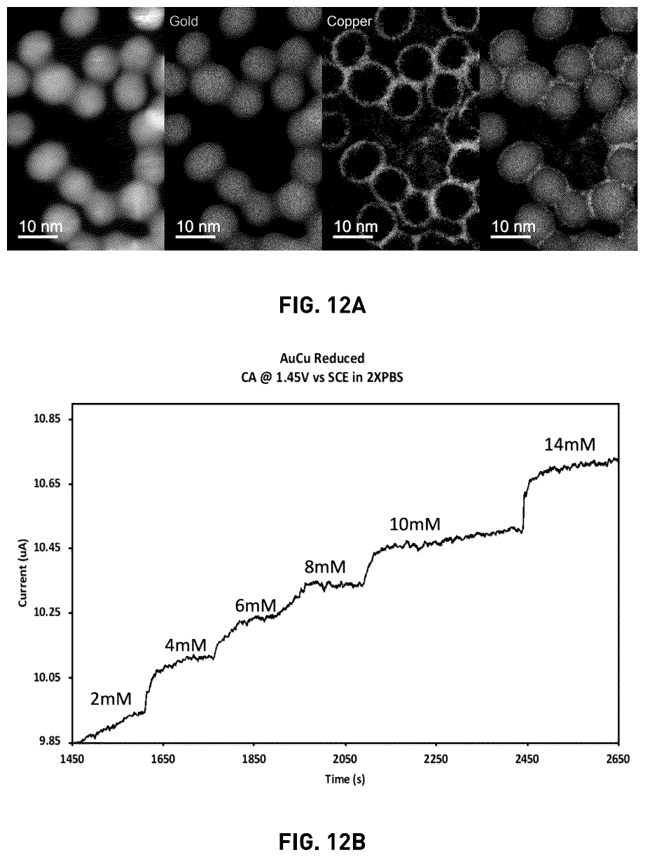

In the illustrated embodiment, the 1:10 Ni:Pt nanocomposite showed greater relative sensitivity to lactate than to glucose and acetic acid. Combining Ni and Pt onto the nanocomposite structure allows the detection of LA is isotonic pH conditions. FIG. 12A illustrates the structure of Copper Gold (Cu Au) NP chains.

FIG. 12A shows the Au-Cu Electron Energy Loss Spectroscopy (EELS). Under electron energy loss spectroscopy, it can be seen that the gold formed the core of the structure, and the copper forms a shell around it (similar to platinum-gold NP chains).

FIG. 12B illustrates real-time detection capabilities of Copper Gold NP chains through Chronoamperometry (CA).

The plotted anodic current at 1.45V vs SCE over time for Au—Cu in 2X PBS (adding lactic acid at regular intervals), indicates increasing current with increasing lactic acid concentration indicates that the Au-Cu nanoparticle chains are also capable of real time lactate detection.

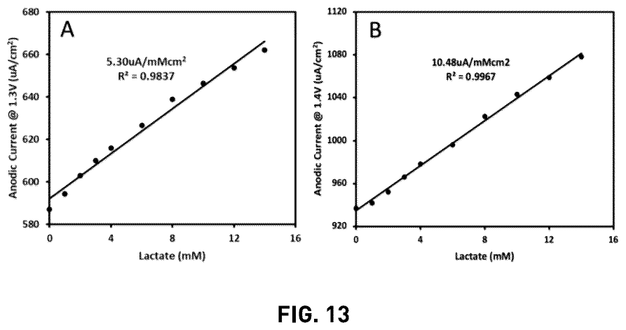

FIG. 13 illustrates an electrochemical analysis (cyclic voltammetry) by Au NP 1:1 Cu:Pt hybrid nanocomposite chain.

Lactate sensitivity at varying oxidation potentials (1.3 V vs. 1.4 V), in 2X PBS buffer for AuNP 1:1 Cu: Pt using CV: The CuPt hybrid demonstrated similar sensitivity to the Ni:Pt hybrid, and at a lower potential (1.3 V compared to 1.5V), and double the sensitivity at 1.4V. By optimizing the surface coverage of active sites of the nanocomposite chains, sensitivity may increase further.

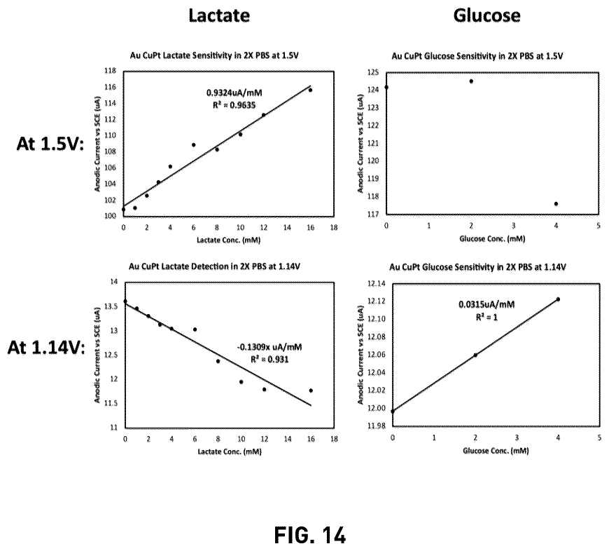

FIG. 14 illustrates methods for Multiple Analyte Detection.

By combining several suitable catalysts and co-catalysts, several analytes can be detected.

This may comprise two separate sensors on a sensor device, or else one sensor with one or more suitable catalysts and co-catalysts, which may detect multiple analytes at different analyte potentials-for example, one sensor may be use to simultaneously detect lactate and glucose: catalysts oxidize lactate and glucose at distinct potentials. A machine learning model may be used to distinguish between analyte signals. In addition to Ni and Pt, Cu and Pt can potentially be used for for LA detection, where preparation methods and ratios can affect performance.

Glucose detection may be observed in many cases at potentials between 0.9 and 1.15V, where LA can be detected at >1.3V. In an embodiment, one sensor may detect both analytes depending on the set potential.

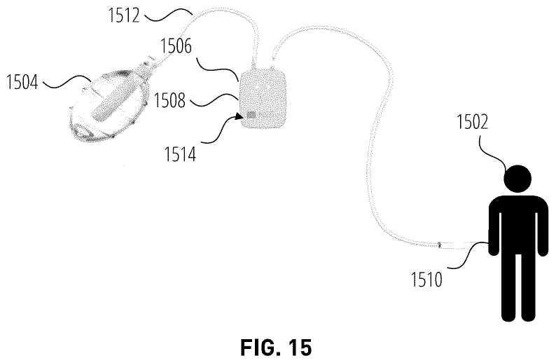

FIG. 15 illustrates an embodiment of the non-enzymatic sensor device 510.

The non-enzymatic sensor device 510 may be integrated into an inline devices 1514 which may have sensors 1506 other than non-enzymatic sensors 1508 (for example, pH, conductivity, etc.). The inline device may be fluidically connected to a patient 1502 via tubing 1512 or a catheter.

Patient 1502 biofluid may flow from a wound drain 1510 (for example), through tubing 1512, over the sensors 1506 and/or non-enzymatic sensors 1508 in the inline device 1514, and to a waste reservoir 1504.

Signals measured by the sensors 1506, 1508, may be stored on a memory of the inline device 1514, or sent to a computer system and/or server, via some connection mechanism (wireless or wired).

In some embodiments, sensors 1506 such as biosensors may be placed on a catheter, and the catheter may be inserted into the body and may allow fluid to be injected into or withdrawn from the body. The catheter can be placed proximal to the surgical site in order to monitor the milieu of the biological fluid proximate to the region. The fluid can be directly sensed locally without the need for negative pressure or it can use negative pressure to assist the fluid to be driven through the catheter. Any number of sensors can be placed on the surface of the catheters such that they are directly in contact with the biological fluid surrounding the area of interest such as the suture line in the case of an anastomosis for example. Sensors may also be placed on the inside of the catheter, a balloon, a pump or any tubing where the fluid can be collected.

In a further embodiment, sensors may be housed within a system that can be placed inline with a catheter. The catheter can be placed proximal to the surgical site in order to monitor the milieu of the peritoneal fluid proximate to the region. The system may be an extension of an existing catheter system. The system may be attached at any time when the catheter is being placed or at a later date.

A lactate sensor may have the following minimum requirements, in accordance with one embodiment:
1. Operate in isotonic pH conditions
2. Real Time, continuous detection
3. Sensitivity: at least 1 mM changes
4. Range: 0.5mmol/L to at least 10 mmol/L
5. Active Lifetime: 5 days following the surgery
6. Lactate Selectivity

Sensors for glucose and other analytes may have the same or similar requirements, with selectivity for the respective analytes.

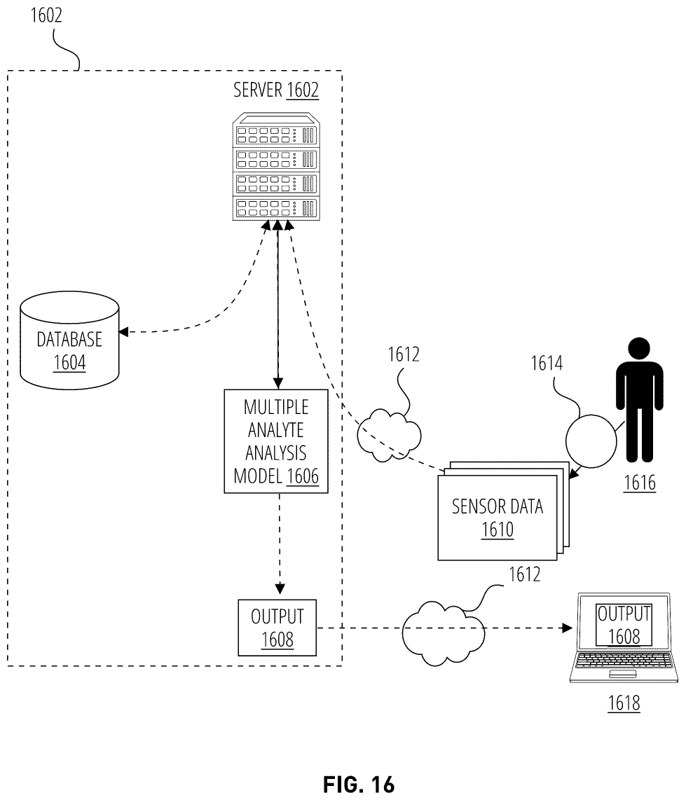

FIG. 16 illustrates an embodiment of the non-enzymatic sensor device 510.

Sensor data 1610, relating to one or more patient 1616 biomarkers, is measured by inline devices 1614, is sent to the server 1602 via a connection mechanism 1612 (wired or wireless), and analyzed by a multiple analyte analysis model 1606, which may be a machine learning (ML) model, trained to either recognize a combination of signals and correlate a combination of signals with a plurality of analyte concentrations, or recognize single signals and extract them from a combination of signals, and subsequently correlate the extracted, single signals, with a plurality of analyte concentrations. Alternatively, or perhaps in combination, the analytes could be identified via the model, based on oxidation/reduction potential, signal/peak location, magnitude, and the like.

The model 1606 may be trained by data on a database 1604, which may comprise sensor data 1610 from a plurality of patients 1616.

The model 1606 generates an output 1608, which may comprise analyte concentration, or some risk assessment relating to a patient's predicted risk of post-surgical complication (such as anastomotic leakage). The output 1608 may be sent to a display system 1618 via a connection mechanism 1612. The display system 1618 may comprise a smart phone, computer, wearable device (i.e. a watch) or the like, which is communicatively coupled to the server 1602.

Sensor data 1610 may be processed by a non-transitory computer-readable storage medium, the computer-readable storage medium including instructions that when executed by a computer, cause the computer to the steps of: receiving at a server, via a sensor device, one or more electrochemical signals; separating, via a multiple analyte analysis model, the one or more electrochemical signals; correlating each of the one or more electrochemical signals to a concentration of each of the one or more analytes; and generating an output based on the concentration of each of the one or more analytes.

Machine learning algorithms or techniques may include, for example, deep learning architectures such as Deep Belief Network (DBN), Stacked Auto Encoder (SAE), Convolutional Neural Network (CNN) or Recurrent Neural Network (RNN) may be used. Other examples include, without limitation, Restricted Boltzmann machines (RBM), Social Restricted Boltzmann Machines (SRBM), Fuzzy Restricted Boltzmann Machines (FRBM), TTRBM models of Deep Belief Networks (DBN) or similar approaches could be used; AE, FAE, GAE, DAE, BAE models of Statistically Adjusted End Use (SAE) models could be used; models such as AlexNet, ResNet, Inception, VGG16, ECNN models of CNN may be used; Bidirectional Recurrent Neural Networks (BIRNN), Long Short-Term Memory (LSTM) networks, Gate Recurrent Unit (GRU) of RNN may also be used. Additional techniques specific to time-series modelling may be employed, including, but not limited to, dynamic time warping, change point detection, and Autoregressive Integrated Moving Average (ARIMA).

In some embodiments, the nanocomposite catalyst template may comprise having gold nanoparticle chains coupled with a carbon support to improve the electron transport of the catalyst.

In some embodiments, data is collected continuously or in designated time intervals.

Further to FIG. 4, the x-ray diffraction (XRD) patterns of nanoparticle (NP) chains and hybrid nanocomposite chains correspond to AuNPs 408, reduced Ni AuNPs 406, reduced Pt AuNPs 404, and reduced 1:3 Ni:Pt AuNP 402 nanocomposite chains.

Further to FIG. 8A, the corresponding lactic acid concentrations shown in plot A and plot C are as follows: 1 mM (802), 5 mM (806), 10 mM (804) and 25 mM (808).

Further to FIG. 8B, plots A and C illustrate graphs corresponding to 6 different lactic acid concentrations: 0 mM, 1 mM, 2 mM, 4 mM, 8 mM, and 16 mM. In plot C, the order of the curve peaks from highest to lowest corresponds to a lactic acid concentration of 16 mM (810), 8 mM (812), 4 mM (814), 2 mM (816), 1 mM (818), and 0 mM (820).

Further to FIG. 12B, the concentrations of lactic acid in different time intervals shown in the plot are the following: 2 mM, 4 mM, 6 mM, 8 mM, 10 mM, and 14 mM.

In some embodiments, electrochemical methods used to detect analytes, such as lactate, comprise differential pulse voltammetry (DPV) and square wave pulses.

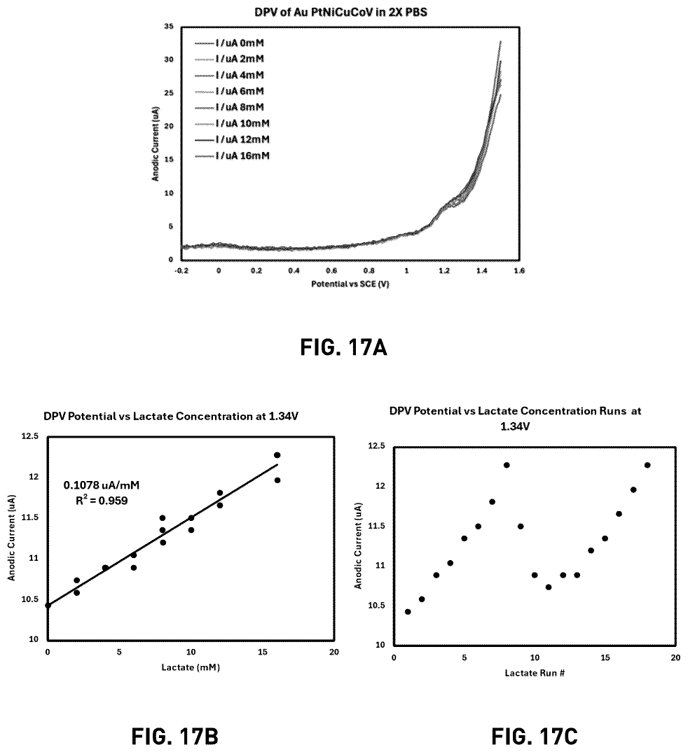

FIG. 17A illustrates a differential pulse voltammetry (DPV) curves using a hybrid nanocomposite chain comprising AuHEA 1/2 PtNiCuCoV. The hybrid nanocomposite chain with the combination of Au, Pt, Ni, Cu, Co, and V appears to be effective in acting as a catalyst to oxidize lactate as the current responses appear to be similar regardless of the concentrations of lactate tested.

FIG. 17B illustrates a differential pulse voltammetry (DPV) plot showing current vs. lactate concentration in the determination of sensitivity of a hybrid nanocomposite chain AuHEA 1/2 PtNiCuCoV, tested in 2X PBS buffer. The plot suggests a strong linear relationship, suggesting that the hybrid nanocomposite chain AuHEA 1/2 PtNiCuCoV demonstrates high sensitivity in detecting lactate.

FIG. 17C illustrates a differential pulse voltammetry (DPV) plot showing current vs. run number in order to demonstrate the hysteresis of the sensor when increasing and decreasing lactate concentrations. In this case, hysteresis was performed with lactate concentrations starting from 0 mM, increasing to 16 mM, decreasing it to 2 mM, and increasing it back to 16 mM. The specific lactate concentrations used in order are the following: 0 mM, 2 mM, 4 mM, 6 mM, 8 mM, 10 mM, 12 mM, 16 mM, 8 mM, 4 mM, 2 mM, 4 mM, 6 mM, 8 mM, 10 mM, 12 mM, and 16 mM.

Relative to the other electrochemical methods described above, DPV demonstrates the most optimal linearity and hysteresis for the data collected.

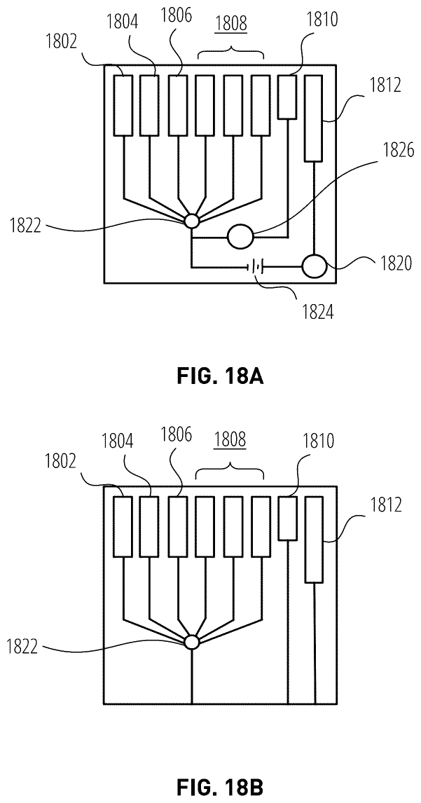

FIG. 18A illustrates a full electrical circuit connecting a plurality of working electrodes, a reference electrode, and a counter electrode. The plurality of working electrodes may comprise working electrodes configured to detect different analytes, such as lactate, glucose and the like. For instance, working electrode A 1802 can be configured to detect lactate at 1.35V while working electrode B 1804 can be configured for glucose at 0.7V. Alternatively, working electrode C 1806 can be configured to have the ability to alternate between lactate and glucose at 1.35V and 0.7V. Working electrodes D, E, and F 1808 can be configured to detect other biological analytes. The working electrodes are connected to a switch 1822 that allows for alternating between the working electrodes within the circuit. The reference electrode 1810 is directly connected to a voltmeter 1826 whereas the counter electrode 1812 is directly connected to an ammeter 1820. The circuit also includes a battery/power supply 1824 that allows the supply of currents between the electrodes.

FIG. 18B illustrates a part of the electrical circuit connecting a plurality of working electrodes (1802, 1804, 1806, 1808), a reference electrode 1810, and a counter electrode 1812 comprising the sensor device assembly. The ammeter 1820, voltmeter 1826, and battery/power supply 1824 as shown in FIG. 18A are part of a larger device, such as an inline device that houses the sensor assembly.

Continuous monitoring of lactate and glucose levels, as well as hormone levels, in the human body may have numerous additional applications outside of post-operative monitoring, including, but not limited to, fitness tracking during or after exercise, glucose monitoring for diabetics, hormone regulation, and the like.

While real-time lactate monitoring can be used to detect ischemia post surgery in patients undergoing GI procedures. A variant of this sensor can be used to monitor athletic performance in real-time. Lactate is secreted in athlete's sweat, and is an indicator of athletic performance. A patch/wearable version of the sensor device can be used to detect real-time lactate measurements which could provide further insight regarding athletic performance and how it is affected in real-time over several shorter periods of time in a more accurate manner than the current standard of measuring lactate changes in larger time increments. For example, by continuously monitoring lactate in a professional hockey game, the staff would have a better idea which players are more exhausted on the field, and can be subbed out early. Monitoring analytes such as uric acid and dopamine, may be of further interest and could readily be monitored using embodiments of the disclosure.

Although lactate and glucose have been described in great detail as target analytes, it should be understood that the systems, methods and devices disclosed herein may be applied to the detection of any group of target analytes where oxidation potentials vary between the target analytes. Simultaneous detection, signal separation, and concentration determination of a variety of target analytes may be aided with machine learning algorithms.

Generally, the processing may be achieved with a combination of hardware and software elements. The hardware aspects may include combinations of operatively coupled hardware components including microprocessors, logical circuitry, communication/networking ports, digital filters, memory, or logical circuitry. The processors may be adapted to perform operations specified by a computer-executable code, which may be stored on a computer readable medium.

The steps of the methods described herein may be achieved via an appropriate programmable processing device or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments discussed above and appreciated by those skilled in the computer and software arts. Appropriate software can be readily prepared by programmers of ordinary skill based on the teachings of the exemplary embodiments, as is appreciated by those skilled in the software arts. In addition, the devices and subsystems of the exemplary embodiments can be implemented by the preparation of application-specific integrated circuits, as is appreciated by those skilled in the electrical arts. Thus, the exemplary embodiments are not limited to any specific combination of hardware circuitry and/or software.

Stored on any one or a combination of computer readable media, the exemplary embodiments of the present invention may include software for controlling the devices and subsystems of the exemplary embodiments, for processing data and signals, for enabling the devices and subsystems of the exemplary embodiments to interact with a human user or the like. Such software can include, but is not limited to, device drivers, firmware, operating systems, development tools, applications software, and the like. Such computer-readable media further can include the computer program product of an embodiment of the present invention for preforming all or a portion (if processing is distributed) of the processing performed in implementations. Computer code devices of the exemplary embodiments of the present invention can include any suitable interpretable or executable code mechanism, including but not limited to scripts, interpretable programs, dynamic link libraries (DLLs), complete executable programs and the like.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Information as herein shown and described in detail is fully capable of attaining the above-described object of the present disclosure, the presently preferred embodiment of the present disclosure, and is, thus, representative of the subject matter which is broadly contemplated by the present disclosure.

What is claimed is:

1. A sensor device for detecting one or more analytes, the sensor device comprising:
one or more working electrodes, each of the one or more working electrodes comprising a plurality of reduced or oxidized self-assembled nanocomposite chains having metal domains deposited on a substrate of each of the one or more working electrodes;
a reference electrode and a counter electrode, connected by a circuit, to the one or more working electrodes.

2. The sensor device of claim 1, wherein each working electrode is configured to measure, via a catalytic reaction between one or more analytes and the reduced or oxidized nanocomposite chains having metal domains, one or more electrochemical signals correlating to a concentration of the one or more analytes.

3. The sensor device of claim 1, further comprising one or more of: polymers or aptamers, functionalized to a surface of the reduced or oxidized nanocomposite chains.

4. The sensor device of claim 1, wherein the metal domains comprise a template and one or more metal ions, the template functionalized by negatively charged capping agents.

5. The sensor device of claim 4, wherein the template is a gold nanoparticle template, and the negatively charged capping agents are citrate ions.

6. The sensor device of claim 4, wherein the one or more metal ions are one or more of: a transition metal and a noble metal.

7. The sensor device of claim 4, wherein the one or more metal ions are selected from a group comprising: Platinum, Nickel, Cobalt, Copper, Manganese, Iridium, Iron, Vanadium, Ruthenium, and Rubidium.

8. The sensor device of claim 1, wherein the sensor device is fluidically coupled to biofluids of a patient for the measurement of the one or more electrochemical signals correlating to the one or more analytes in the biofluids of the patient.

9. The sensor device of claim 1, wherein the one or more analytes comprise at least one of glucose or lactate.

10. The sensor device of claim 1, wherein the sensor device is configured to operate in isotonic and physiological pH conditions.

11. A method for detecting and monitoring one or more analytes, the method comprising:
measuring, via a catalytic reaction between the one or more analytes and the plurality of reduced or oxidized nanocomposite chains having metal domains deposited on one or more working electrodes of a sensor device, one or more electrochemical signals of the one or more analytes.

12. The method of claim 11, further comprising:
receiving at a server, via the sensor device, the one or more electrochemical signals;
separating, via a multiple analyte analysis model, the one or more electrochemical signals;
correlating each of the one or more electrochemical signals to a concentration of each of the one or more analytes; and
generating an output based on the concentration of each of the one or more analytes.

13. The method of claim 11, wherein the sensor device is fluidically coupled to biofluids of a patient, for measuring the one or more electrochemical signals relating to the one or more analytes in the biofluids of the patient.

14. The method of claim 13, wherein the one or more electrochemical signals is measured continuously and in real-time.

15. The method of claim 12, wherein the one or more analytes comprises at least one of glucose or lactate.

16. The method of claim 11, wherein the measurement of the one or more electrochemical signals is conducted in isotonic and physiological pH conditions.

17. The method of claim 11, wherein the one or more analytes are identified, via the multiple analyte analysis model, based on oxidation potential or reduction potential of the one or more analytes.

18. The method of claim 12, wherein the output comprises a post-surgical complication risk assessment of the patient.

19. A non-transitory, computer readable storage medium, the computer-readable storage medium including instructions that when executed, performs the steps of:
receiving at a server, via a sensor device, one or more electrochemical signals;
separating, via a multiple analyte analysis model, the one or more electrochemical signals;
correlating each of the one or more electrochemical signals to a concentration of each of the one or more analytes; and
generating an output based on the concentration of each of the one or more analytes.

20. The non-transitory, computer readable storage medium of claim 19, wherein the one or more analytes comprises at least one of glucose or lactate.
